SEQUENCE LISTING (4 sequences)

SEQ ID NO: 1 - DNA, 24 bp, Artificial Sequence
  Synthetic primer
catggtcattttaagtgcctcatc

SEQ ID NO: 2 - DNA, 21 bp, Artificial Sequence
  Synthetic primer
tagtgaaagcgaacttcagtg

SEQ ID NO: 3 - DNA, 23 bp, Artificial Sequence
  Synthetic primer
atccatgatcgcctggtccattc

SEQ ID NO: 4 - protein, 230 aa, Artificial Sequence
  Synthetic peptide
MDPNTVSSFQVDCFLWHVRKRVADQELGDAPFLDRLRRDQKSLRGRGSTLGLDIETATRAGKQIVERILKEESDEALKMTMASVPASRYLTDMTLEEMSRDWSMLIPKQKVAGPLCIRMDQAIMDKNIILKANFSVIFDRLETLILLRAFTEEGAIVGEISPLPSLPGHTAEDVKNAVGVLIGGLEWNDNTVRVSETLQRFAWRSSNENGRPPLTPKQKREMAGTIRSEV

(19) United States
(12) Patent Application Publication
(10) Pub. No.: US 2011/0268764 A1
(43) Pub. Date: Nov. 3, 2011

(54) IMMUNE RESPONSE INDUCING PREPARATIONS

(75) Inventors: Monika Sachet, Vienna (AT); Michael Bergmann, Klosterneuburg (AT); Thomas Muster, Vienna (AT); Andrej Egorov, Vienna (AT)

(73) Assignee: AVIR GREEN HILLS BIOTECHNOLOGY RESEARCH DEVELOPMENT TRADE AG, Vienna (AT)

(21) Appl. No.: 13/099,810

(22) Filed: May 3, 2011

Related U.S. Application Data
(63) Continuation of application No. 12/063,388, filed on Feb. 8, 2008, now abandoned.

(30) Foreign Application Priority Data
Aug. 8, 2005 (AT) A1332/2005

(51) Int. Cl. A61K 39/00; A61K 39/02; A61K 39/21; A61P 37/04; A61K 39/04; A61K 39/29; A61K 39/13; C12N 5/07; A61K 39/12; A61K 39/145

(52) U.S. Cl. 424/208.1; 424/184.1; 424/277.1; 424/204.1; 424/234.1; 424/274.1; 424/265.1; 424/209.1; 424/207.1; 424/248.1; 424/225.1; 424/217.1; 435/375

(57) ABSTRACT

The present invention provides a pharmaceutical composition with an adjuvant based on an apathogenic virus, together with an antigen. The adjuvant has a natural or through genetical engineering no, reduced or altered expression of an endogenous interferon antagonist or endogenous immune suppressor.

This application is a national phase application under 35 U.S.C. §371 of International Application No. PCT/AT2006/000335 filed 8 Aug. 2006, which claims priority to Austrian Patent Application No. A 1332/2005 filed 8 Aug. 2005. The entire text of each of the above-referenced disclosures is specifically incorporated herein by reference without disclaimer.

The present invention relates to pharmaceutical compositions comprising an antigen.

A vaccine is used to prepare a human or animal's immune system to defend the body against a specific pathogen, usually a bacterium, a virus or a toxin. Depending on the infectious agent to prepare against, the vaccine can be a weakened bacterium or virus that lost its virulence, or a toxoid, a modified, weakened toxin or particle from the infectious agent. The immune system recognizes the vaccine particles as foreign, reacts to and remembers them. During contact with the virulent version of the agent the immune cells are prepared to counter the foreign substances and neutralizing the agent. Live but weakened virus vaccines are used against rabies, and smallpox; killed viruses are used against poliovirus and influenza; toxoids are known for diphtheria and tetanus.

The influenza virions consist of an internal ribonucleoprotein core (a helical nucleocapsid) containing the single-stranded RNA genome, and an outer lipoprotein envelope lined inside by a matrix protein (Ml). The segmented genome of influenza A virus consists of eight molecules (seven for influenza C) of linear, negative polarity, single-stranded RNAs which encode 11 polypeptides, including: the RNA-dependent RNA polymerase proteins (PB2, PB1 and PA) and nucleoprotein (NP) which form the nucleocapsid; the matrix membrane proteins (Ml, M2); two surface glycoproteins which project from the lipid containing envelope: hemagglutinin (HA) and neuraminidase (NA); the nonstructural protein NS1, the nuclear export protein (NEP) and the proapoptitic protein PB1-F2. Transcription and replication of the genome takes place in the nucleus and assembly occurs via budding on the plasma membrane. The viruses can reassort genes during mixed infections.

Influenza virus adsorbs via HA to sialyloligosaccharides in cell membrane glycoproteins and glycolipids. Following endocytosis of the virion, a conformational change in the HA molecule occurs within the cellular endosome which facilitates membrane fusion, thus triggering uncoating. The nucleocapsid migrates to the nucleus where viral mRNA is transcribed. Viral mRNA is transcribed by a unique mechanism in which viral endonuclease cleaves the capped 5′-terminus from cellular heterologous mRNAs which then serve as primers for transcription of viral RNA templates by the viral transcriptase. mRNA transcripts terminate at sites 15 to 22 bases from the ends of their templates, where oligo (U) sequences act as signals for the addition of poly (A) tracts. Of the eight viral RNA molecules so produced, six are monocistronic messages that are translated directly into the proteins representing HA, NA, NP and the viral polymerase proteins, PB2, PB1 and PA. The other two transcripts undergo splicing, each yielding two mRNAs which are translated in different reading frames to produce Ml, M2, NS1, NEP. In other words, the eight viral RNA segments code for eleven proteins: nine structural and two nonstructural.

Dendritic cells (DC) are the most potent antigen presenting cells (APC) and are capable to induce immune responses to foreign microbial antigens but also to self-antigens. The latter is relevant for the induction of anti-tumour immune responses. Viruses are very potent activators of DCs, since a major task of DCs is to combat viral infection. For this reason, viruses or virus related structures might be used as immunostimulatory adjuvant for vaccine purposes. An example of a virus family with high proinflammatory capacities are influenza A viruses. Infection of human DCs with this virus stimulates a strong proliferative and cytotoxic immune response against viral antigens (Bhardwaj, N. et al. J Clin Invest., 94:797-807, 1994). The immunostimulatory capacity of influenza A virus infection even leads to the induction of a strong T-cell immunity to a non-immunogenic protein, when co-administered with the virus (Brimnes et al., J Ex Med 198(1), 2003: 133-144).

US 2004/0109877 A1 and WO 99/64068 describe attenuated viruses, which have a modified interferon (IFN) antagonist activity. IFNs are substances which invoke an antiviral state in target cells. One example therein refers to influenza viruses with a partially mutated NS1 gene or a complete knock-out of the NS1 gene (the virus is also referred to as “delNS1” or “NS1/99”). The IFN antagonist activity allows the virus to proliferate in a cell while bypassing the cells innate immunity based on IFNs by either inhibiting the activity or the production of IFN and is therefore responsible for the pathogenicity of the virus. Mutations or knock-outs of NS1 generally result in an increase of IFNs and therefore in lower virus multiplication. The increase in the IFN concentration also has antiviral effects against other viruses. Therefore, the use of such attenuated viruses as a vaccine has been suggested against a broad range of viruses and antigens. Further uses therein include the introduction of foreign antigens into the attenuated virus by recombinant methods.

NS1 (FIG. 7; amino acid sequence: NCBI database acc. nr.: MNIV1; NS1 nucleotide sequence: NCBI database acc. no.: J02150) has been shown to be an antagonist of type I IFN (Garcia-Sastre, A. et al. Virology., 252:324-30, 1998), NF-κB (Wang, J Virol. 74(24): 11566-11573 (2000)) and the interferon induced double stranded RNA activated kinase PKR (Bergmann, M. et al. J Virol., 74:6203-6, 2000).

The alpha/beta interferon (IFN-α/β) system is a major component of the host innate immune response to viral infection (Basler et al., Int. Rev. Immunol. 21:305-338, 2002). IFN (i.e., IFN-β and several IFN-α types) is synthesized in response to viral infection due to the activation of several factors, including IFN regulatory factor proteins, NF-κB, and AP-1 family members. As a consequence, viral infection induces the transcriptional upregulation of IFN genes. Secreted IFNs signal through a common receptor activating a JAK/STAT signaling pathway which leads to the transcriptional up-regulation of numerous IFN-responsive genes, a number of which encode antiviral proteins, and leads to the induction in cells of an antiviral state. Among the antiviral proteins induced in response to IFN are PKR, 2′,5′-oligoadenylate synthetase (OAS), and the Mx proteins (Clemens et al., Int. J. Biochem. Cell Biol. 29:945-949, 1997; Floyd-Smith et al., Science 212:1030-1032, 1981; Haller et al., Rev. Sci. Technol. 17:220-230, 1998).

It was shown that the 230 amino acid (aa) comprising NS1 protein comprises a RNA binding domain from amino acids 1-73. NS1 is capable to bind snRNA, poly(A) and dsRNA as a dimer and has a further effector domain at the carboxy-end for regulating cellular mRNA processing (Wang et al., RNA 5 (1999): 195-205).

US 2003/0157131 and WO 99/64571 suggest the use of an attenuated influenza A virus with an interferon-inducing phenotype containing a knockout of the NS1 gene segment as a vaccine, administered prior to wild-type influenza infections. These viruses are only capable of replication in an interferon-free environment.

WO 99/64570 describes methods to grow NS1 deficient influenza A and B viruses in interferon deficient environments, e.g. embryonated chicken eggs below the age of 12 days, or cell lines deficient in IFN production like Madin-Darby canine kidney (MDCK) cells or VERO cells.

The number of adjuvants currently approved for human application is very limited and is practically restricted to aluminium salts and MF59 (Singh et al., Nature Biotech. 17: 1075-1081, 1999). Although many more immunostimulating compounds, like oil in water emulsions, are known, their application is limited by side effects like toxicity (e.g. the cancerogenous Freund's adjuvant). Therefore the development of or search for adjuvants for the application in humans, mammals, other animals or even cell cultures is necessary for diverse applications.

The present invention provides a pharmaceutical composition for inducing a specific immune response against an antigen, comprising

(a) said antigen and

(b) an adjuvant, which is an apathogenic virus.

Many viruses have evolved mechanisms to counteract the host IFN response and, in some viruses, including vaccinia virus, adenovirus, and hepatitis C virus, multiple IFN-antagonist activities have been reported (Beattie et al., J. Virol. 69:499-505, 1995; Brandt et al., J. Virol. 75:850-856, 2001; Davies et al., J. Virol. 67:1688-1692, 1993; Francois et al., J. Virol. 74:5587-5596, 2000; Gale et al., Virology 230:217-227, 1997; Kitajewski et al, Cell 45:195-200, 1986; Leonard et al., J. Virol. 71:5095-5101, 1997; Taylor et al., Science 285:107-110, 1998; Taylor et al., J. Virol. 75:1265-1273; 2001). An apathogenic virus in a composition according to the present invention does not contain an (active) IFN antagonist, either naturally or by removal with genetic methods. The apathogenic virus to be used according to the invention is, of course, not pathogenic, i.e. it does not impose a virus infection burden on the individual receiving the pharmacological composition according to the present invention. Methods for removing activity of IFN antagonists in viruses are for example disclosed (in general) in Sambrook et al. (Molecular Cloning: A Laboratory Manual (Cold Spring Harbor Laboratory, New York, 1989), Ausubel et al. (Current Protocols in Molecular Biology, Greene Publishing Assoc. and Wiley and Sons, New York, 1994) and the like. Among negative-strand RNA viruses, several different IFN-subverting strategies have been identified that target a variety of components of the IFN system. The influenza virus NS1 protein, for example, prevents production of IFN by inhibiting the activation of the transcription factors IFN regulatory factor 3 and NF-κB and blocks the activation of the IFN-induced antiviral proteins PKR and OAS (Bergmann et al., J. Virol. 74:6203-6206, 2000; Garcia-Sastre et al., Virology 252:324-330, 1998; Talon et al., J. Virol. 74:7989-7996, 2000; Wang et al., J. Virol. 74:11566-11573, 2000). Among the paramyxoviruses, different mechanisms are employed by different viruses (Young et al., Virology 269:383-390, 2000). For example, the “V” proteins of several paramyxoviruses have previously been shown to inhibit IFN signaling, but the targets of different V proteins vary (Kubota et al., Biochem. Biophys. Res. Commun. 283:255-259, 2001; Parisien et al., Virology 283:230-239, 2001). In the case of Sendai virus, the “C” proteins, a set of four carboxy-coterminal proteins, have been reported to block IFN signaling both in infected cells and when expressed alone (Garcin et al., J. Virol. 74:8823-8830, 2000; Garcin et al., J. Virol. 73:6559-6565, 1999; Gotoh, FEBS Lett. 459:205-210, 1999; Kato et al., J. Virol. 75:3802-3810, 2001; Komatsu et al., J. Virol. 74:2477-2480, 2000). In contrast, respiratory syncytial virus, which encodes neither a C nor a V protein, produces two nonstructural proteins, NS1 and NS2, that are reported to cooperatively counteract the antiviral effects of IFN (Bossert et al., J. Virol. 76:4287-4293, 2002; Schlender et al., J. Virol. 74:8234-8242, 2000). Ebola virus, a nonsegmented, negative-strand RNA virus of the family Filoviridae that possesses a genome structure similar to that of the paramyxoviruses (Klenk et al., Marburg and Ebola viruses, p. 827-831. in R. G. Webster and A. Granoff (ed.), Encyclopaedia of virology, vol. 2. Academic Press, New York, N.Y., 1994), also encodes at least one protein, VP35, that counteracts the host IFN response (Basler et al., Proc. Natl. Acad. Sci. USA 97:12289-12294, 2000). The present invention provides the use of the apathogenic virus as adjuvant. An adjuvant is used to increase the immune reaction towards an antigen. Therefore, in particular, the antigen and the adjuvant are different or separate moieties, i.e. the adjuvant is not or does form a part of the antigen.

Viral IFN antagonists have been shown to be important virulence factors in several viruses, including herpes simplex virus type 1, vaccinia virus, influenza virus, and Sendai virus. Analysis of viruses with mutations in genes encoding herpes simplex virus type 1 ICP34.5 (Chou et al., Science 250:1262-1266, 1990; Markowitz et al., J. Virol. 71:5560-5569, 1997), vaccinia virus E3L (Brandt et al., J. Virol. 75:850-856, 2001), influenza virus NS1 (Garcia-Sastre et al., Virology 252:324-330, 1998; Talon et al., Proc. Natl. Acad. Sci. USA 97:4309-4314, 2000), and Sendai virus C (Durbin et al., Virology 261:319-330, 1999; Garcin et al., Virology 238:424-431, 1997) proteins has demonstrated an important role for each of these IFN antagonists in viral pathogenicity in mice. Because IFN antagonists are important virulence factors, their identification and characterization should provide important insights into viral pathogenesis.

Viruses, which are apathogenic in humans, which naturally do not contain an PKR or IFN antagonist and which might therefore be used as an adjuvant are reovirus (Stong et al., EMBO J. 1998 Jun. 15; 17(12):3351-62) and VSV (Stojdl et al., J. Virol. 2000 October; 74(20):9580-5.) Moreover, examples of viruses, which are apathogenic in humans and which have a deleted IFN antagonist the Newcastle disease virus lacking the V protein (Huang et al., J. Virol. 2003 August; 77(16):8676-85). In a preferred embodiment of the composition according to the invention the apathogenic virus is selected from apathogenic vaccinia virus, adenovirus, hepatitis C virus, Newcastle disease virus, paramyxoviruses, Sendai virus, respiratory syncytial virus, Filoviridae, herpes simplex virus type 1, reovirus, influenza virus or VSV.

Even more preferred is a composition according to the invention, wherein the apathogenic virus is a genetically engineered virus comprising a mutation, a truncation, a knock-out or a reduced expression of a viral endogenous interferon antagonist gene or endogenous immune suppressor gene (which is present in the wild type, or deposited variant of the specific virus). These mutations lead to an apathogenic phenotype of the virus and enhance the immune response via the induction of cytokines. This was shown for influenza virus (Ferko et al. J. Virol 2004, Stasakova et al J. Gen. Virol. 2004).

Examples of attenuated viruses lacking the IFN (or PKR) antagonist which can be used as vaccine adjuvants are following: i) The herpes virus Myb34.5 (Nakamura et al. J Clin Invest 2002, 109:871), which lacks the PKR antagonist. ii) The vaccinia virus MVA (Modified virus Ankara) which lacks EL3 protein (Hornemann et al., J Virol. 2003, August; 77(15):8394-407). Therefore preferably the virus in a composition according to the invention is selected from herpes virus Myb34.5, vaccinia virus MVA or Newcastle disease virus lacking the V protein.

In another preferred embodiment the composition comprises as an adjuvant a genetically engineered influenza virus comprising a mutated or truncated NS1 protein, or a knockout or a reduced expression of the NS1 gene segment.

Preferably the expression of the NS1 protein is at least 5 fold, preferably at least 10 fold, lower compared to a wild type virus. The reduced expression of NS1 is generally enough to abolish function of NS1.

Preferably the reduction of NS1 expression is achieved by mutations in the 3′ terminal and/or 5′ non-coding nucleotides of the segment 8, preferably by mutations in the NS1-ORF, further preferred by replacing the non-coding sequences of segment 8 with non-coding regions of the NA segment. Reduction of NS1 is achieved by mutations in the 3′ terminal and 5′ noncoding nucleotides of the Segment 8. Moreover, reduced expression can be achieved by modifying the expression of the NS1-ORF. E.g., the NS1 ORF is expressed after the ORF of the NS2 in segment using a stop-start sequence for bicistonic messages. The non-coding regions of the NA segment of the virus can be used to replace the non-coding sequences of segment 8. Moreover the non-coding region of segment 8 of influenza B virus can be used. Random mutations are also possible after an analyse to their effects.

In the composition of the present invention the adjuvant, which is a modified influenza virus, enhances the immune response of said antigen. As an antigen any type of substance can be used against which an immune reaction in the animal or cell culture is desired. Such antigens are for example parts of pathogenic organisms such as different viruses, bacteria, or fungi. The term “different viruses” herein refers to viruses other than the genetically engineered virus which forms the adjuvant. By the mutation, truncation knock-out or reduced expression of an interferon or immune suppressor, which would be normally produced by the wild-type virus, the virus is highly reduced in its pathogenicity and capability to cope with an immune system of a host or immune response of an adequate cell culture.

Preferably in the composition according to the invention the adjuvant is a genetically engineered influenza virus comprising a mutated or truncated NS1 protein, or a knockout or a reduced expression of the NS1 gene segment. The NS1 protein is a good target as influenza endogenous interferon (IFN) antagonist. Such a genetically altered influenza virus shows highly reduced pathogenicity to the extend that it is hard to cultivate in normal tissue cells in the presence of interferon, thus having an attenuated phenotype.

One example of the adjuvant of the present invention is the delNS1 virus. The delNS1 is an influenza derived strain, which lacks the open reading frame of the non-structural protein NS1. It has been proven in prior art to simulate several indicators of immune responses such as interferons (IFN), NF-κB, PKR and other cytokines of the innate immune response (Ferko et al, J Virol 2004, above). Type I IFN has been implicated in the maturation of dendritic cells and in the priming of antigen specific CD8+ and CD4+ T-cell response. NF-κB is a central key protein in the immune response. Activation of PKR is thought to be advantageous for breaking immunological tolerance, a problem which abrogates the immune response against endogenous tumour associated antigens (Leitner, W. et al. Nat Med., 9:33-9, 2003). Therefore delNS1 virus is specifically appropriate to stimulate DCs. Although IFNs inhibit delNS1 virus proliferation, the immune-enhancing effect is not diminished in the time frame of an application as adjuvant.

Based on the efficacy of the delNS1 the present invention preferably provides a composition as defined above, wherein the genetically engineered influenza virus contains a deletion of the entire NS1 gene segment.

Another preferred embodiment of the present invention is a composition as described above, wherein the genetically engineered influenza virus contains a truncated NS1 protein with a C-terminal deletion, while retaining less than the first 40, 50 or 60, especially 70 or 80, in particular 90, 100, 110, 120, 124 or 126 amino acids of the wild-type NS1 gene product. Such modifications of the NS1 protein constitute phenotypes, which are intermediates of a virus with a fully functional NS1 protein and the delNS1. The NS1 protein contains an RNA binding site from amino acids 1 to 73 (Wang et al., RNA 5 (1999): 195-205), and a C-terminal effector function regulating cellular mRNA processing. Mutants comprising deletions in this region are specially impeded in their functionality. The RNA binding capacity of the NS1 protein relates to the interferon susceptibility of the virus. All these mutant viruses, comprising NS1 mutations in the range from a complete NS1 deletion (delNS1) to a only 126 amino acid containing NS1 protein, can grow in media with very little IFN, such as 8-12 day old embryonated chicken eggs. The viruses show a sufficiently low virulence for an application as adjuvants without endangering the patient, animal or cell culture.

The present invention includes also the use of naturally occurring mutant influenza viruses A or B having truncated NS1 proteins in a composition according to the invention. For influenza A viruses, these include, but are not limited to: viruses having an NS1 of 124 amino acids (Norton et al., 1987, Virology 156: 204-213).

For influenza B viruses, these include, but are not limited to: viruses having an NS1 truncation mutant comprising 127 amino acids derived from the N-terminus (B/201) (Norton et al., 1987, Virology 156: 204-213), and viruses having a NS1 truncation mutant comprising 90 amino acids derived from the N-terminus (B/AWBY-234) (Tobita et al., 1990, Virology 174: 314-19). The present invention encompasses the use of naturally occurring mutants analogous to NS1/38, NS1/80, NS1/124, (Egorov, et al., 1998, J. Virol. 72 (8): 6437-41) as well as the naturally occurring mutants, A/Turkey/ORE/71, B/201 or B/AWBY-234.

Therefore, a preferred composition according to the invention comprises the genetically engineered influenza virus containing the NS1-124 mutation, which only contains the N-terminal 124 amino acids of the NS1 protein, i.e. the sequence of the amino acids 1 to 124 of the NS1 protein, as disclosed in NCBI database acc. no.: MNIV1.

Another preferred composition according to the invention is a composition as described above, wherein the genetically engineered influenza virus contains the NS1-80 mutation, which only contains the N-terminal 80 amino acids of the NS1 protein, i.e. the sequence of the amino acids 1 to 80 of the NS1 protein. The efficacy of these influenza mutants as an adjuvant is given in the examples.

A specially preferred composition according to the invention, wherein the NS1 protein of the genetically engineered influenza virus lacks a functional RNA binding domain. The RNA binding domain of a wild type influenza NS1 protein is defined as the first 73 N-terminal amino acid. Although RNA binding is relatively unspecific and a lack of a significant amount of amino acids or RNA binding elements would be sufficient to prevent RNA binding, several key amino acids have been identified by the absence of one such key amino acids the NS1 protein is rendered incapable of RNA binding. Such amino acids are for example Arg38 (or R38) and Lys41 (or K41) in the NS1 protein of influenza A. An example of a NS1 protein in the scope of the present invention would be a NS protein lacking the C-terminal part and retaining a N-terminal part of less than 41 of amino acids. Viral RNA is an effective stimulator of antigen presenting cell. Thus binding and masking of these RNAs by the RNA binding domain of NS1 protein reduces the immune response. A loss in one of these key amino acid residues renders the mutant fragment of NS1 inoperable and thus incapable of interfering in cellular RNA related processes such as RNA processing. An influenza virus with such a mutation shows very low virulence and an attenuated phenotype.

Further genetic modification to create a virus with low virulence target regulating non-coding sequences of the NS1 gene, are disclosed in Bergmann et al., Virus Res. 1996 September; 44(1):23-31 or Muster et al., Proc Natl Acad Sci USA. 1991 Jun. 15; 88(12):5177-81. This method allows to construct a virus with low levels of NS1 protein expression but leaving the NS1 open reading frame intact.

In a further preferred composition according to the invention the virus is an attenuated virus. Attenuated viruses are obtained by procedures that weaken a virus and render it less vigorous and do not cause an illness. Mutations in the NS1 as described above attenuate the virus by themselves if no other virulence factors are introduced, which can compensate the loss of an effective wild type NS1 gene product and the possibility for virus reversions is eliminated. Such attenuated viruses can be used in vaccine formulations.

Further genetic modification to create a virus with low virulence target regulating non-coding sequences of the neuraminidase (NA) gene, as is disclosed in Bergmann et al., Virus Res. 1996 September; 44(1):23-31. An example for modification of NA 3′ and 5′ noncoding sequence is the replacement of NA 3′ and 5′ noncoding sequences by NS1 3′ and 5′ noncoding sequences (Muster et al., Proc Natl Acad Sci USA. 1991 Jun. 15; 88(12):5177-81). Such a modified virus is attenuated and immunogenic. Therefore the present invention also relates to a composition as defined above, wherein the genetically engineered influenza virus is attenuated by replacing the non-coding sequences of the neuraminidase (NA) gene by non-coding sequences of the NS1 gene or other genetic modifications of the virus. A further preferred attenuated influenza virus used in the composition according to the invention is attenuated by replacing the non-coding sequence of the NS1 gene by those of other gene segments.

Preferably, in the composition according to the invention the influenza virus is an influenza A virus or influenza B virus. Nowadays it is common practice to modify these influenza strains by reverse genetics and growth of these strains can be well handled on special media which are known to the artisan.

According to the present invention in the composition as defined above the antigen is admixed to the virus. This enables the artisan to easily prepare a composition according to the invention for any desired application right before application. Accordingly, the adjuvant can be stored separately from the antigen against which immunity is desired. This type of preparation is well effective as is shown in the examples.

Another composition according to the invention provides the antigen complexed or covalently linked to the genetically modified virus. The advantage in this preparation lies in that both compounds can be treated in one step, e.g. they can be simultaneously assayed if a determination of the antigenic and viral load is to be determined or they can be simultaneously purified by chromatographic techniques.

A further preferred composition according to the invention comprises at least one additional adjuvant. Such additional adjuvants further augment the immune response against the antigen and are for example aluminium salts, microemulsions, lipid particles, oligonucleotides such as disclosed by Singh et al., (Singh et al. Nature Biotech. 17: 1075-1081, 1999).

Therefore, the present invention relates to a composition as defined above, wherein the at least one additional adjuvant is selected from mineral gels, aluminium hydroxide, surface active substances, lysolecithin, pluronic polyols, polyanions or oil emulsions, or a combination thereof. Of course the selection of the additional adjuvant depends on the intended use. The application of cheap but toxic adjuvants is for example not advised for certain animals, although the toxicity may depend on the destined organism and can vary from no toxicity to high toxicity.

Another preferred embodiment of the composition of the present invention further comprises buffer substances. Buffer substances can be selected by the skilled artisan to establish physiological condition in a solution of the composition according to the invention. Properties like pH and ionic strength as well as ion content can be selected as desired.

A further preferred composition according to the invention, comprises a pharmaceutically acceptable carrier. The term “carrier” refers to a diluent, e.g. water, saline, excipient, or vehicle with which the composition can be administered. For a solid composition the carriers in the pharmaceutical composition may comprise a binder, such as microcrystalline cellulose, polyvinylpyrrolidone (polyvidone or povidone), gum tragacanth, gelatine, starch, lactose or lactose monohydrate; a disintegrating agent, such as alginic acid, maize starch and the like; a lubricant or surfactant, such as magnesium stearate, or sodium lauryl sulphate; a glidant, such as colloidal silicon dioxide; a sweetening agent, such as sucrose or saccharin.

In a preferred composition according to the invention the antigen is selected from tumour antigens or antigens of infectious pathogens like different viruses, bacteria, parasites or fungi. Generally, even compounds that are not antigenic by themselves, i.e. do not provoke an immune response by B- and T-cells in an organism, an organism or cultures of immune cells can be immunised against these compounds with a use in a composition according to the present invention.

Even more preferred is a composition as described above, wherein the antigen is selected from gp160, gp120 or gp41 of HIV, HA and NA of influenza virus, antigens of endogenous retroviruses, antigens of human papilloma viruses, especially E6 and E7 protein, melanoma gp100, survivin, Her2neu, NY-ESO, tuberculosis antigens, hepatitis antigens, polio antigens, etc.

A preferred composition according to the invention may further comprise a cytokine in order to modulate the immune response. It is for example possible with the selection of appropriate cytokines to stimulate either CD4+ T-cells for a primarily humoral, i.e. antibody mediated, immune response or CD8+ T-cells for a cellular mediated immune response or to attract DCs.

Another embodiment is to express an immunostimulatory cytokine within the virus or delNS1 virus. This can be accomplished by genetic manipulation of the virus, e.g. by introducing an oligonucleotide coding for said cytokine into the virus. A composition according to the invention may therefore comprise a virus with a genetic sequence for an immunostimulatory cytokine.

The present invention also provides a method for the manufacture of a composition according to the invention comprising the step of admixing the antigen with the virus comprising a mutation, a truncation, a knock-out or a reduced expression of an endogenous interferon antagonist gene or endogenous immune suppressor.

The present invention also relates to a pharmaceutical formulation for ingestion, comprising a composition as described above and a suitable carrier. Such a pharmaceutical formulation presents the pharmaceutical composition according to the invention in a form suitable for delivery or application. Suitable solid carriers for ingestion are well known for the skilled artisan and some examples are given above. Therapeutic formulations suitable for oral administration, e.g. tablets and pills, may be obtained by compression or moulding, optionally with one or more accessory ingredients. Compressed tablets may be prepared by mixing the constituent(s), and compressing this mixture in a suitable apparatus into tablets having a suitable size. Prior to the mixing, the composition may be mixed with a binder, a lubricant, an inert diluent and/or a disintegrating agent and further optionally present constituents may be mixed with a diluent, a lubricant and/or a surfactant. Alternatively the composition according to the invention can be formulated in liquid form for oral application. Thus, the pharmaceutical composition may be formulated as syrups, capsules, suppositories, powders, especially lyophilised powders for reconstitution with a carrier for oral administration, etc. Such a formulation can further contain a stabilising agent or a preservative.

A further aspect of the invention is a pharmaceutical formulation for intranasal delivery, comprising a composition as defined above and a suitable carrier in the form of nasal drops or for intranasal delivery by a spray device. Nasal drops can be easily used and constitute a practical way to administer the composition of the present invention.

Another embodiment of the invention is a pharmaceutical formulation for subcutaneous, intramuscular, intravascular or intraperitoneal injection, comprising a composition as defined above and a suitable stabilising carrier. Injections provide a way of entry, which guarantees the application of the pharmaceutic formulation according to the invention and can be used to for systemic application of the adjuvants.

The present invention also provides a method for the manufacture of a pharmaceutical formulation as defined above comprising the step of admixing a composition according to the invention with a suitable carrier.

A further aspect of the present invention is the use of an apathogenic virus, preferably an attenuated NS1 deficient influenza A virus, as described above, as an immune modulating adjuvant to induce an immune-enhancing effect of an antigen or to overcome pathogen induced immunosuppression or cancer induced immunosuppression or for the preparation of such an immune modulating adjuvant. Accordingly the NS1 deficient influenza A virus, which either lacks the NS1 gene or has severe mutations/truncations in the NS1 gene or reduced NS1 expression as described above (which is understood under “NS1 deficiency”), can be administered to a living being or a cell culture together with an antigen in order to enhance the immune reaction against the antigen. Of course other apathogenic viruses as mentioned above may also be used. Especially together with the use of cancer antigens it is possible to stimulate immune cells to become reactive against cancerous cells, thus overcoming a cancer induced immunosuppression. Such treated immune cells, e.g. dendritic cells, T cells or B cells, can be either treated in vivo or ex vivo and reintroduced in to a living being. Accordingly, a pathogen induced immunosuppression can be overcome by using an antigen of the pathogen.

The present invention also provides a method for in vitro activation of dendritic cells with a specific antigen characterized in that dendritic cells are contacted in vitro with a composition comprising an antigen and an apathogenic as adjuvant as described above. Dendritic cells can be obtained from cell cultures or from living beings and made reactive to the antigen by method known in the art (s. Sambrook et al., above, Ausubel et al., above) and the methods given below in the examples. The apathogenic virus provides a further stimulant to improve the conditioning of the dendritic cells. Preferably the dendritic cells are immature dendritic cells. These cells are characterized by high endocytic activity and low T-cell activation potential. Dendritic cells constantly sample the surroundings for viruses and bacteria. Once they have come into contact with such an antigen, they become activated into mature dendritic cells. Such dendritic cells can activate T-helper cells to promote an immune response in a cell culture including these cells or a living being.

Preferably the contacting of the composition according to the invention and the dendritic cells is carried out for 10 minutes to 8 hours, preferably for 10 to 60 minutes.

In another preferred method the specific antigen used for contacting is an isolated tumour or virus antigen, a recombinant tumour or virus antigen or a tumour or virus lysate. With these antigens the dendritic cells can be made reactive to several molecular entities comprised by these antigens or antigenic substances. The virus lysate is preferably obtained through infection of tumor cells with the apathogenic virus, preferably the NS1 deficient influenza virus, as described above.

Furthermore the present invention provides dendritic cells obtainable according to a method described above. Such dendritic cells can be used to stimulate T-helper cells in a cell culture or in a patient against a specific antigen.

The present invention is described in more detail with the help of the following examples and figures to which it should, however, not be limited.

### FIGURES

FIG. 1: Immature monocyte derived DCs are cocultured with autologous T cells after infection with delNS1 or NS1-124 (m.o.i.=2). Pictures are taken 12 h and 24 h after infection. Staining against nucleoprotein (NP) of influenza (green) and cell nucleus DNA (red) is shown. There are apoptotic bodies seen in all infected DCs (arrows), no difference between the two viruses is detectable. Staining was done with two different donors cells; one experiment is shown.

FIG. 2: Annexin V staining of immature monocyte derived DCs 5 h after infection with delNS1, NS1-124 or PR8 (m.o.i.=2) for detection of phosphatidylserin-switch as an early marker of apoptosis. grey histogram: mock infected cells; open histogram: virus infected cells. One representative experiment from six different donors is shown.

FIG. 3: (A) Surface marker expression of immature monocyte derived DCs 30 h after infection with delNS1, NS1-124 and PR8 (m.o.i.=2). The intensity of staining with the indicated antibodies is shown; mock infected cells (grey histogram), delNS1 (-) NS1-124 (- - -) and PR8 ( [symbol] ). Results were obtained after analysis of at least 10 000 cells. One representative experiment from six different donors is shown.

(B) Surface marker expression of immature monocyte derived DCs 24 h after transfection with total vRNA or incubation with viral protein. The intensity of staining with the indicated antibodies is shown; mock infected cells (grey histogram), vRNA transfected cells ( [symbol] ) and cells pulsed with viral protein (- - -). Results were obtained after analysis of at least 10 000 cells. One representative experiment from three different donors is shown.

FIG. 4: Effect of delNS1 or NS1-124 infection of MODCs, which were pulsed with tumour cell lysates on the induction of anti-tumour cytotoxic immune response. As a tumour lysate Panc1 cell disrupted by freeze-thaw method were taken. T cells were twice stimulated by pulsed DCs and infected. CTL assay were done by standard Europium assay. Cytotoxicity was assessed using (A) a specific target (Panc1) or (B) an unspecific target (K-562 cells) T−: T-cells not stimulated with DCs. T+ Panc1: T-cell stimulated with DCs primed with tumour lysate; T+ Panc1+delNS1: T-cell stimulated with DCs primed with Panc1 lysate and then infected with delNS1 virus. T+ Panc1+NS-124: T-cell stimulated with DCs primed with Panc1 lysate and then infected with NS-124 virus. The results in percentage specific lysis represent the mean of triplicate measurements. Representative results from one of four experiments from different donors

FIG. 5: Effect of virus-induced tumour cell lysis on the stimulation an antitumour immune response. Virolysates were obtained using delNS1 or NS1-124 for lysis of Panc1 cells. Virolysates or conventional oncolysates were used to pulse MODCs. T cells were twice stimulated by pulsed DCs pulsed with oncolysate or virolysate. CTL assays were done by standard Europium assay. Cytotoxicity was assessed using (A) a specific target (Panc1) or (B) an unspecific target (K-562 cells). T−: T-cells not stimulated with DCs. T+ Panc1: T-cell stimulated with DCs primed with conventional tumour lysate; T+ Panc1delNS1: T-cell stimulated with DCs primed with Panc1 lysate obtained by infection with delNS1 virus. T+ Panc1NS1-124: T-cell stimulated with DCs primed with Panc1 lysate obtained by infection with NS-124 virus. The results in percentage specific lysis represent the mean of triplicate measurements. Representative results from one of four experiments from different donors.

FIG. 6: Six mice per group were immunised i.p. with trivalent (H1,H3,B) influenza inactivated vaccine antigens (vaccine) in a dose of 15 or 5 μg per animal alone or in combination with 6.5 log A/PR/8/34 or delNS influenza live viruses. Three weeks later serum samples were tested in ELISA for the presence of antibodies (IgG) against the influenza B component. Admixing of inactivated viral antigen with the live virus enhanced the production of antibodies at least 4 times in the delNS group. This effect was prominent in both groups of mice inoculated with different doses of inactivated vaccine. At the same time the immune adjuvant effect of A/PR/8/34 virus was much weaker and detected only in the group of animals receiving high dose of inactivated vaccine.

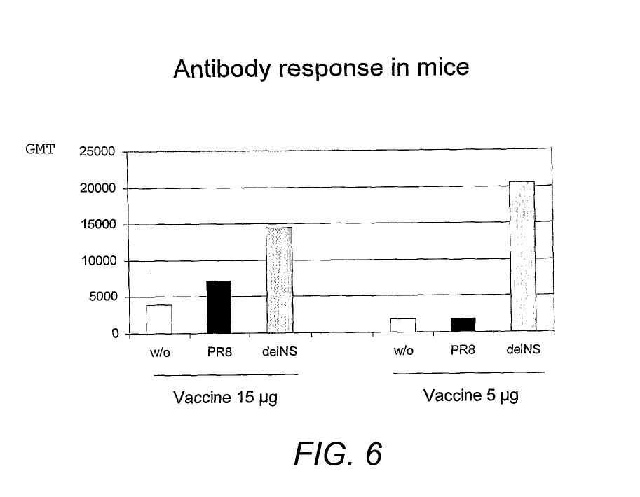

FIG. 7: Amino acid sequence of nonstructural protein NS1 of the influenza A virus (strain A/PR/8/34) (SEQ ID NO: 4).

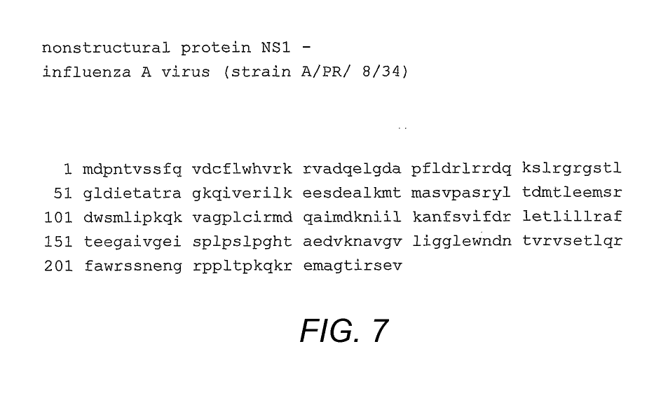

### EXAMPLES

The presented examples provide results of the immunostimulatory capacity of monocyte derived dendritic cells (MODC)s, after treatment with NS1-deletion or NS1-truncation viruses. It is demonstrated that the NS1 modified viruses induce a potent cytokine response in these cells and even improve dendritic cell maturation. Moreover delNS1 infection of DC stimulated with tumour cell lysate relates to an enhanced cytotoxic T-cell response, which is specific for tumour related antigens.

### Example 1

### Materials and Methods

### Cells and Viruses:

Functional DCs can be generated ex vivo from peripheral blood monocytes or from bone marrow derived cells. For tumour vaccination DCs are stimulated ex vivo with defined HLA-restricted tumour-associated antigens (TAA) or with a lysate of tumour cells (oncolysates) and subsequently reinfused or reinjected into the tumour bearing patient. Clinical phase I trials revealed that this type of immunotherapy is feasible and associated with little side effects in humans. However, response rates in first clinical phase I trial were only observed in rare cases. Yet it is a major challenge to improve efficacy of DC based vaccination.

Human pancreas cell line Panc1 (ATCC) and the human erytroleukemia cell line K562 were cultured in RPMI 1640 medium (GibcoLife Technologies, USA) containing 10% fetal calf serum (PCS) and supplemented with 5 mg/ml gentamicin. Vero (ATCC) cell adapted to grow on serum-free medium were maintained in serumfree OPTIPRO medium (Invitrogen). Influenza A/PR/8 (PR8) virus and NS1 deletion viruses were generated as described using the helper virus based transfection system, i.e. the open reading frame of the NS1 gene is deleted (Egorov, A. J Virol., 72:6437-41, 1998). PR8 wt virus contains a transfected NS wt gene segment and encodes a wild-type NS1 protein of 230 amino acids. The delNS1 virus contains a complete deletion in the NS gene segment (Garcia-Sastre, A., Virology., 252:324-30, 1998); NS1-80 and NS1-124 are PR8 derived mutants which only code the N-terminal 80 and 124 amino acids (aa) of the NS1 protein. The plasmid coding for the NS1-80 NS segment was constructed using a plasmid coding for segment 8 transcribed by a poll promoter and the primer pair 3′NS269: 5′CATGGTCATTTTAAGTGCCTCATC-3′ (SEQ ID NO: 1) and 5′NS-TRG-415: 5′TAGTGAAAGCGAACTTCAGTG-3′ (SEQ ID NO: 2). The plasmid coding for the NS1-124 segment was constructed using the above mentioned plasmid of coding for segment 8 of wild type virus and the primer pairs 3′NS400T: 5′ atccatgatcgcctggtccattc-5′ (SEQ ID NO: 3) and 5′NS-TRG-415. For propagation of the viruses, Vero cells were infected at a multiplicity of infection (m.o.i.) of 0.1 and cultured in OPTIPRO medium containing 5 μg/ml trypsin (Sigma) at 37° C. for 2-3 days. Virus concentrations were determined by plaque assays on Vero cells.

Viral Infection and Replication; Generation of vRNA and Whole Viral Protein; Transfection of Viral RNA:

Monocyte derived dendritic cells (MODC) were washed with PBS and infected with PR8-wt and NS1 deletion viruses at an m.o.i. of 0.1. After incubation for 30 min, the inoculum was removed, cells were washed with PBS, overlaid with OPTIPRO medium containing 2.5 μg/ml trypsin (Sigma) and incubated at 37° C. for 48 h. Supernatants were assayed for infectious virus particles in plaque assays on Vero cells. vRNA was obtained from virus purified by centrifugation. RNA was isolated by QIA-Amp RNA extraction kit (Quiagen) according the manufacture's protocol. For transfection of viral RNA, 5 day old immature MODC were incubated with RNA complexed with lipofectamin for 2 hours following a medium change. Total viral protein was extracted by standard methods from virus purified by centrifugation. Grade of purification was determined by SDS gel electrophoresis. Amount of viral protein was determined by Bradford analysis.

### Isolation and Generation of Immature and Mature DCs:

Peripheral mononuclear cells (PBMC) were obtained by standardised gradient centrifugation with Ficoll-Paque (Pharmacia, Uppsala, Sweden) from 100 ml of EDTA whole blood. Thereafter, CD14 positive cells were separated by magnetic sorting using VARIOMACS technique (Miltenyi BiotecGmbH, Bergisch Gladbach, Germany) according to the manufacturer's protocol. Isolated CD14+ cells were cultured at a concentration of 1×10⁶ cells/ml in standard culture flasks (Costar, Cambridge; MA) for 5 days in RPMI1640 medium (GibcoLife Technologies, USA) containing 10% fetal calf serum (PCS) and supplemented with 5 mg/ml gentamicin at 37° C. in a humidified 5% CO₂ atmosphere in the presence of 1000 U/ml of each, recombinant human (rh) granulocyte-macrophage colony stimulating factor (GM-CSF) (Leukomax; AESCA, Traiskirchen, Austria) and rh interleukin-4 (IL-4) (PBH, Hannover, Germany). On day 2, rh GM-CSF and rh IL-4 were again added to the cultures at a concentration of 1000 U/ml.

### Preparation of Tumour and Viro Lysate:

Generation of oncolysates: Panc1 (approximately 10⁸ cells) were washed twice with PBS, after they have been dissolved from the flask and in 2 ml PBS lysed by five freeze and thaw cycles. Generation of virolysates: Tumour cells were infected with Influenza A (PR8 or delNS1) with a m.o.i.=1 and cultured in RPMI1640. 16 hours later cells were dissolved from the flask and lysed in 2 ml PBS by five freeze and thaw cycles. The protein concentration was determined according to Bradford.

### Preparation of T Cells and Co-Culture:

PBMCs were prepared as above. CD14 neg. charge was separated with magnetic beads in a fraction CD3+. This fraction was used for co-culture. 1×10⁶ pulsed DCs were mixed with 5×10⁶ T-cells (CD3+) in RPMI 1640 medium containing 10% fetal calf serum (PCS) and supplemented with 5 mg/ml gentamicin (GibcoLife Technologies, USA) for 7 days.

### Stimulation of Virus Infected MODC to Induce a CTL Response:

Immature DCs were incubated with tumour lysate obtained by the freeze/thaw procedure as described in the material and methods. Thereafter immature DCs were infected with delNS1 and NS1-124 virus at a m.o.i.=0.5. DCs were then co-cultured with peripheral blood T-cells. The level of specific T-cell stimulation was then determined in a Europium assay against the specific target Panc-1.

Stimulation of MODC with Virolysate Versus Oncolyate to Induce a CTL response:

The immature DCs thus obtained were pulsed with tumour lysate or virolysate (100 ug/ml) on day 5, which again was washed out after 12 hours. Thereafter, the culture was washed and incubated for 36 hours in RPMI 1640 containing 1000 ng/ml TNF-alpha to promote DC maturation for 24 hours. 4 hours before co-culture 1,000 U/ml IFN-gamma (Imukin®) and IPS (Alexis Cooperation, Lausen, Switzerland) was added. DCs were then co-cultured with peripheral blood T-cells. The level of specific T-cell stimulation was then determined in a Europium assay against the specific target Panc-1 or against K-562 cells, respectively.

### Repulsing:

1×10⁶ fresh pulsed DCs were mixed with 5×10⁶ T-cells from the co-culture. Co-culture cells were washed twice with medium before mixing with fresh DCs.

### Flow Cytometric Analysis:

The phenotype of immature and mature dendritic cells was determined by single or two-colour fluorescence analysis. Cells (3×10⁵) were resuspended in 50 μl of assay buffer (PBS, 2% PCS and, 1% sodium azide) and incubated for 30 min at 4° C. with 10 μl of appropriate fluorescein isothiocyanate (FUC) or phycoerythrin (PE)-labelled mAbs. After incubation, the cells were washed twice and resuspended in 500 μl assay buffer. Cellular fluorescence was analysed in an EPICS XL-MCL flow cytometer (Coulter, Miami, Fla., USA). 10000 events were acquired for each sample and the percentage of positive cells was reported. Monoclonal antibodies specific for human IgG1Isotyp, CD3, CD14, CD80, CD86, CD83, MHC class I, MHC class II (Immunotech, Vienna, Austria) and CD40 (PharMingen, PD) were used to characterise DCs. For detection of apoptosis Annexin V Apoptosis Detection Kit (Genzyme Diagnostics) was used. Immature DCs were infected with delNS1 or PR8 at a m.o.i.=2. 5 h p.i. cells were washed and stained to detect phosphatidylserin exposure at the outer leaflet of the cell membrane as an early apoptotic marker according to the manufacturer's instructions. Apoptotic cells were detected and quantified by flow cytometry.

### Immunofluorescent Staining:

Cells were fixed on slides via cytospin and fixed with 3.7% paraformaldehyde for 10 min at room temperature. Slides were washed five times with PBS and permeabilised with 0.5% Triton X for 20 min. Primary mouse antibody against nucleoprotein (NP) of Influenza A (R.u.P. Margaritella) was used at a dilution of 1:100 in PBS with 1% bovine serum albumin and incubated for 1 h at room temperature. Cells were washed with PBS and incubated with Alexa® Fluor 488 donkey antimouse IgG (Molecular Probes, Eugene, Oreg.) and propidium iodide, for nucleus staining, for 1 h at room temperature. Cells were washed again with PBS and mounted with SlowFade° Light (Molecular Probes) and sealed. Pictures were taken with a Zeiss LSM 510 confocal microscope.

### Cytotoxicity Assay (Europium Release Assay):

The generated CD8 positive T-lymphocytes were first isolated as described above and then co-cultured with tumour/viro lysate pulsed autologous DCs for 5-7 days without any cytokines. Thereafter their functional properties were tested by a standardised Europium release assay in regard of their ability to specifically lyse. For this purpose, 5×10⁶ target cells (Panc1) were labelled by europium. The labelled target cells were mixed with allogenic T-lymphocytes (effector cells) at a ratio of 50:1 to 3:1. After 4 hours of incubation at 37° C. the remnant was analysed in a Delfia fluorometer (Victor 2, Wallac, USA) for determination of the released quantity of europium. The percentage of lysis was calculated as follows: (experimental release−spontaneous release)/(total release−spontaneous release)×100. As control targets for NK cells K562 cells were used.

Cytokine Detection after Infection with Influenza A PR8 and delNS1:

Immature DCs were infected with virus (m.o.i.=2) and cultured with and without autologous CD3 positive T-lymphocytes at a ratio of 1:5 for 24 hours in RPMI1640 medium containing 10 fetal calf serum (PCS). Supernatant was then screened for TNF-alpha, IL-10, IL-6 (DPC Immulite, Los Angeles, USA), IL-2, IL-4, IL-12(p70), IFN-gamma (Upstate, USA), IFN-alpha and IFN-beta (ELISA Kit, PBL Biomedical Laboratories).

### Example 2

### Induction of Cytokines by NS1 Deletion Mutants

Since it was intended to investigate the effect of NS1 deletion viruses as an adjuvant, we analysed initially the cytokines response, which was induced by the viruses in professionally antigen presenting cells such as dendritic cells. First, immature monocyte derived dendritic cells (MODC)s of 4 different probands with NS1 deletion virus delNS1, NS1-124 or PR8 wild type virus (m.o.i.=2) were infected. The delNS1 contains no NS1 protein at all and is a replication deficient virus (Garcia-Sastre et al., 1998). The NS1-124 is an attenuated NS1 mutant and contains the N-terminal 124 aa of the NS1. The virus induced induction of cytokines 24 hours after infection (Table 1) was determined. Despite the activity of cytokines is complex and frequently cannot be narrowed to a single function, the focus in this assay was on cytokines of the innate “unspecific” immune response such as TNF, IL-6, type I IFN (IFN-alpha), since the function of immature DCs is to be activated and activates the immune system at the onset of an infection. Polarising cytokines of the specific immune system such as IL-10, IFN-gamma and IP-10 were also included. IL-10 is associated with the induction of a strong B-cells immune response. IFN-gamma and IP-10 direct the immune system towards a cytotoxic T-cell. Infection of DCs with both NS1 deletion viruses induced a massive cytokine response of all pro-inflammatory cytokines of the innate immune system (TNF, IL-6, IFN-alpha) as compared to non-infected dendritic cells. Importantly, the induction of these cytokines was significantly higher by the NS1 deletion viruses as compared to the wild type virus (4-100 fold). The stimulation of IFN-alpha tended to be slightly higher for the delNS1 virus as compared to the NS1-124 virus. Not unexpectedly there are high interindividual differences for virus cytokines stimulation. Polarising cytokines such as IFN gamma or IL-10 were not induced in the immature MODCs. This might not be surprising since the induction of a polarised T-cell response is not the function of immature dendritic cells. However, IP10 was well induced by both deletion viruses.

TABLE 1
Cytokine production in virus infected DCs

| | | Virus | | | |
|---|---|---|---|---|---|
| Cytokine* | Proband | delNS1 | NS1-124 | PR8 | non infected |
| TNF-alpha | 1 | 2220 | 2061 | 234 | 11 |
| | 2 | 4571 | 4591 | 665 | 46 |
| | 3 | 5897 | 5353 | 1284 | 21 |
| | 4 | 4363 | 2978 | 212 | 23 |
| IL-6 | 1 | 1467 | 1275 | 541 | 9 |
| | 2 | 1049 | 849 | 85 | 0 |
| | 3 | 332 | 112 | 59 | n.d. |
| | 4 | 670 | 565 | 124 | n.d. |
| IL-10 | 1 | 0 | 0 | 0 | 0 |
| | 2 | 0 | 0 | 0 | 0 |
| | 3 | 0 | 0 | 6 | n.d. |
| | 4 | 0 | 0 | 0 | n.d. |
| IFN-alpha | 1 | 1689 | 698 | 12 | 0 |
| | 2 | 737 | 525 | 0 | 0 |
| | 3 | 711 | 520 | 0 | 0 |
| | 4 | 3385 | 1882 | 176 | 0 |
| IFN-beta | 1 | 64 | 165 | | 187 |
| | 2 | 730 | 373 | | n.d. |
| | 3 | 513 | 597 | | n.d. |
| IFN-gamma | 1 | 0 | 0 | | 0 |
| | 2 | 0 | 0 | | n.d. |
| | 3 | 0 | 0 | | n.d. |
| | 4 | 0 | 0 | | n.d. |
| IP10 | 1 | 342 | 466 | | 163 |
| | 2 | 561 | 821 | | n.d. |
| | 3 | 639 | 529 | | n.d. |
| | 4 | 1383 | 826 | | n.d. |

*concentrations in pg/ml

Further analysis determined, whether single viral component such as whole viral RNA or whole viral protein could account for cytokine stimulation. For this assay it was chosen to analyse TNF and IFN-alpha since this cytokines were stimulated most potent by the mutant viruses (Table 2). Neither whole viral RNA nor whole viral protein could account for a cytokine response, which was significant higher than non-infected cells, despite whole virus protein had a tendency to be little higher than control. These data indicate that whole virus but not single components have to be present for cytokine activation pattern observed during virus stimulation.

TABLE 2
Cytokine production in immature MODCs transfected with
vRNA/incubated with viral protein/infected with virus

| | TNF* | IFN-alpha* |
|---|---|---|
| mock transfected | 13.8 | 0 |
| viral RNA (6 μg transfected) | 28.5 | 0 |
| viral whole protein (50 μg) | 6 | 0 |
| delNS1(moi = 2) | 4262 | 1707 |

*concentrations in pg/ml

The main function of dendritic cells is to activate lymphocytes. Therefore the cytokine profile of infected MODCs in co-cultivation with CD8 positive lymphocytes was analysed. Focus was on the T-cell subset since these cells are specifically important for the induction of an anti-tumour immune response. 5-day old immature MODCs were used to be able to compare the results with the assay described above. In the co-culture experiment the main known polarising cytokines were included, which promote stimulation of T-cell such as IL-4 and IL-10 (stimulation of Th-2 cells) and IL-2 and IFN-gamma (stimulation of Th-1 cells). The cytokine response of non-infected DCs, which are co-cultured with T-cell are slightly higher than the cytokine response of non-infected immature DCs alone (Table 3). It is hypothesised that this low cytokine response already signifies DC activation. Again, viral infection was associated with a massive increase in cytokine response as compared to non-infected co-cultured dendritic cells. In this assay a third delNS1 mutant virus with an intermediate deletion (NS1-80) was included. This virus was shown to induce solid T-cell immune responses in the animal. In contrast to virus stimulation of immature dendritic cells co-cultured virus infected dendritic cells produce substantial levels of IFN gamma but also low levels of IL-2 and IL-10. Interestingly, IFN-alpha was significantly higher than in immature DCs. Other cytokine of the innate immune system (TNF, IL-6) were equally induced in viral infected co-culture as compared to immature DC cultures. Thus, the cytokine profile suggests that viral infecting strongly activates DCs and supports a cytotoxic T-cell directed immune response.

TABLE 3
Cytokine production in virus infected DCs co-cultured
with T-cells

| | | Virus | | | |
|---|---|---|---|---|---|
| Cytokine * | Proband | delNS1 | NS1-80 | NS1-124 | non-infected |
| TNF-alpha | 1 | 3501 | 1902 | 2503 | n.d. |
| | 2 | 1732 | 1028 | 1919 | 50 |
| | 3 | 889 | 919 | 919 | n.d. |
| | 4 | 3456 | 1171 | 3881 | n.d. |
| IL-2 | 1 | 48 | 83 | 51 | 0 |
| | 2 | 63 | 107 | 73 | n.d. |
| | 3 | 70 | 131 | 103 | n.d. |
| | 4 | 99 | 250 | 181 | n.d. |
| IL-4 | 1 | 0 | 0 | 0 | 0 |
| | 2 | 0 | 0 | 0 | n.d. |
| | 3 | 0 | 0 | 0 | n.d. |
| | 4 | 0 | 0 | 0 | n.d. |
| IL-6 | 1 | 538 | 306 | 446 | 52 |
| | 2 | 1133 | 518 | 630 | n.d. |
| | 3 | 156 | 144 | 163 | 38 |
| | 4 | 2848 | 935 | 2146 | n.d. |
| IL-10 | 1 | 49 | 50 | 51 | 16 |
| | 2 | 65 | 52 | 61 | n.d. |
| | 3 | 22 | 27 | 22 | n.d. |
| | 4 | 299 | 331 | 300 | n.d. |
| IL-12 p70 | 1 | 0 | 0 | 0 | 0 |
| | 2 | 0 | 0 | 0 | n.d. |
| | 3 | 0 | 0 | 0 | n.d. |
| | 4 | 0 | 0 | 0 | n.d. |
| IFN-alpha | 1 | 4790 | 5623 | 5494 | 77 |
| | 2 | 6342 | 5442 | 5942 | n.d. |
| | 3 | 3506 | 4516 | 4162 | n.d. |
| | 4 | 1414 | 1467 | 1502 | n.d. |
| IFN-beta | 1 | 226 | 241 | 284 | 54 |
| | 2 | 162 | 189 | 186 | n.d. |
| | 3 | 145 | 58 | 137 | n.d. |
| IFN-gamma | 1 | 384 | 507 | 406 | 0 |
| | 2 | 396 | 445 | 417 | n.d. |
| | 3 | 788 | 763 | 902 | n.d. |
| | 4 | 483 | 617 | 512 | n.d. |

* concentrations in pg/ml

### Example 3

### NS1 Deletion Mutant Abortively Infect and Induce Apoptosis in MODC

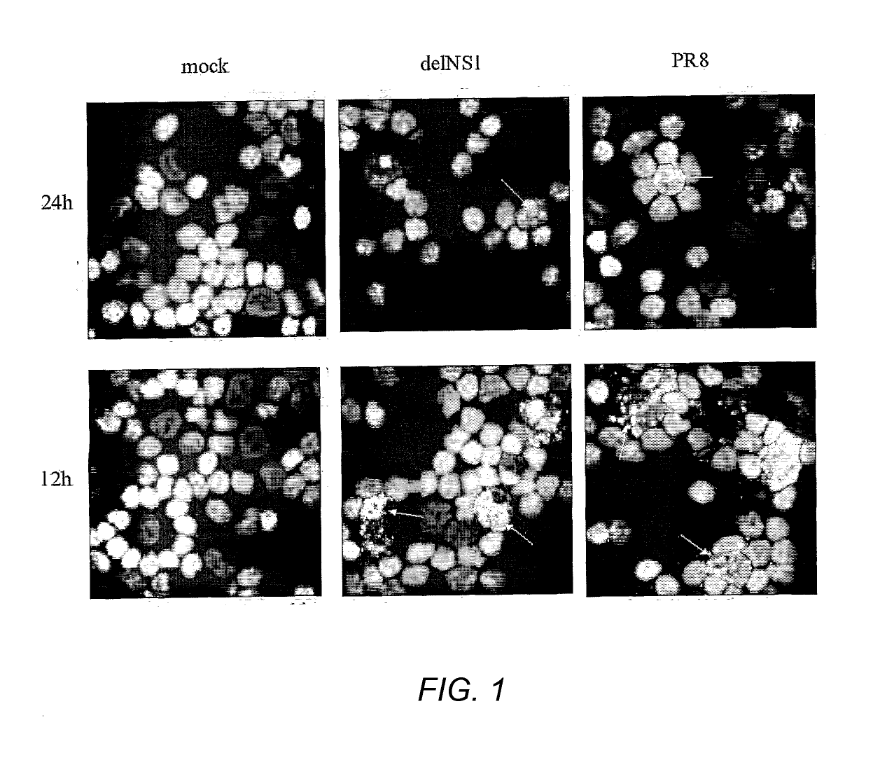

In order to analyse whether virus replicates in MODC, cells were infected with either delNS1 of NS1-124. The generation of viral protein was the determined by immunohistochemistry. This experiment was done in the co-culture system using DC and autologous CD8 positive T-cells to mimic the situation in a lymph node. Positive staining for viral proteins was observed for DCs infected with delNS1 virus and for DCs infected with NS1-124 virus (FIG. 1). Interestingly, T-cell adhered on virus infected DCs in a rosette-like configuration. This phenomenon was not observed in uninfected DCs. This might be explained by expression of HA by DCs and adherence of lymphocytes. Alternatively it could be due to the interaction of activated lymphocytes with DCs.

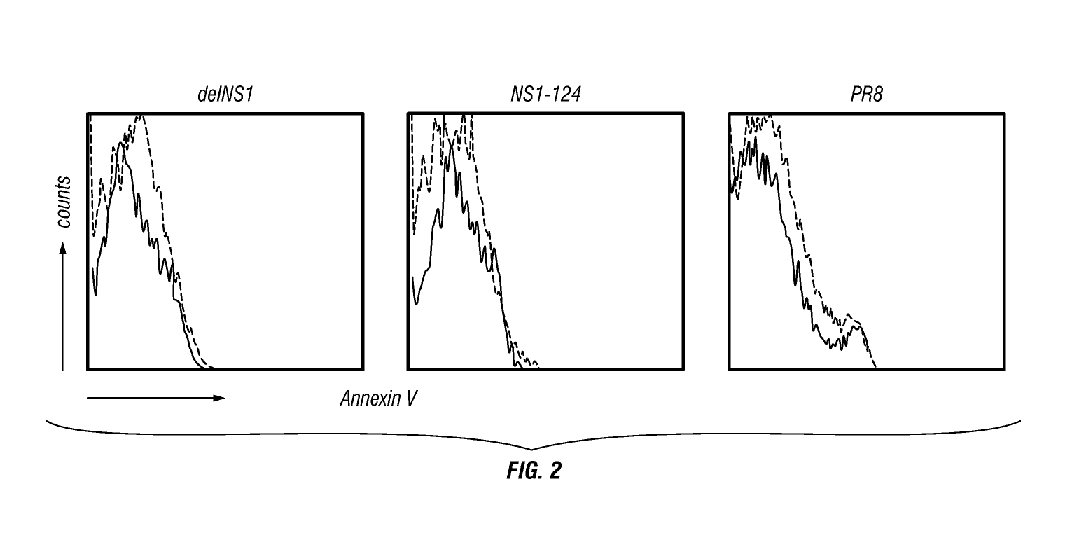

Virus induced complete cytopathic effect (CPE) was usually observed in DCs after 24-48 hours. CPE corresponded to apoptosis as determined by Annexin V staining (FIG. 2). In supernatant of delNS1 deletion virus infected MODC no infectious titers are found, indicating that infection is not productive and that MODCs are abortively infected by NS1 deletion mutants.

### Example 4

### Induction of Maturation Marker by NS1 Deletion Marker

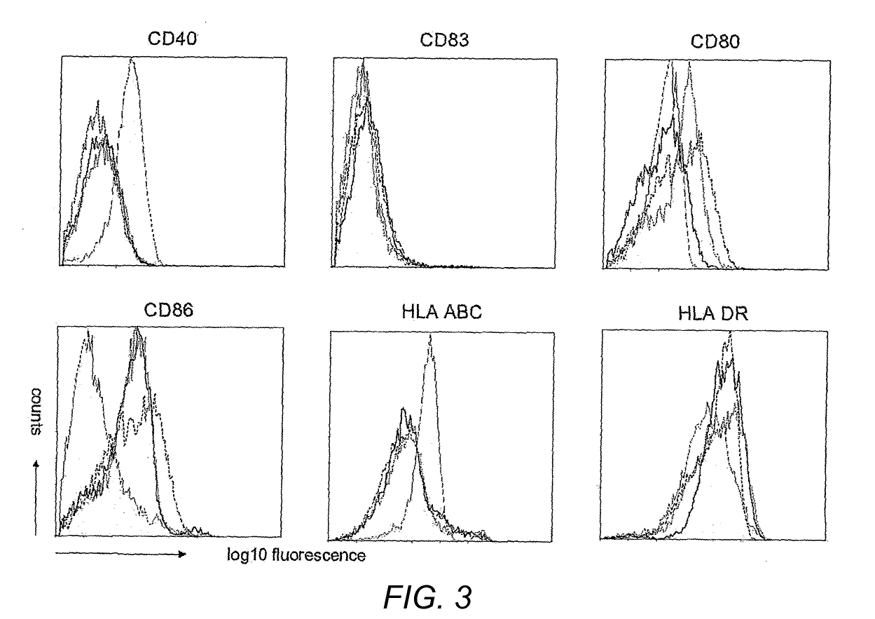

The ability of delNS1 deletion mutants delNS1 and NS1-124 to induce maturation in dendritic cells was estimated. Therefore the virus induced increase of maturation markers CD83, CD80 and CD86 on the surface of immature dendritic cells was determined in addition to the expression or surface expression on MODCs such as CD40, MHC-class I and MHC-class II. 5 different probands were analysed. Despite of interindividual differences there was a clear expression profile. FIG. 3a shows a representative experiment. Again there was no difference for both deletion viruses. Highest induction (approximately 10 fold) was observed for CD86, and MHCII. Low induction was seen for CD83 and CD80. CD40 and MHC class I were slightly downregulated in immature MODC. Downregulation for these molecules relevant for antigen presentation in immature MODC corresponds to the inability of these cells to induce polarising cytokines such as IL-2 or IFN gamma as discussed above (Table 1).

It was further analysed, whether subcomponents of the virus such as whole viral RNA or whole virus protein accounts for the induction of the surface markers (FIG. 3b). Subcomponents of the virus showed a different picture than the whole virus. The transfection of total viral RNA transfected into immature MODC induced high levels of CD86 and CD40 and low levels of CD83, CD80, MHC class I and HMC class II. The transfection procedure alone did not have any effect on these molecules. Whole virus protein reduced the expression of CD40, CD86, HLA class I, HLA class II and had no effect on CD83 and CD80. Thus, viral protein might account for the virus-mediated effect on CD40 and MHC class I. Viral RNA might account for virus induced induction of maturation markers.

### Example 5

### DelNS1 Virus Infection of MODC Enhances its Immunostimulatory Capacity

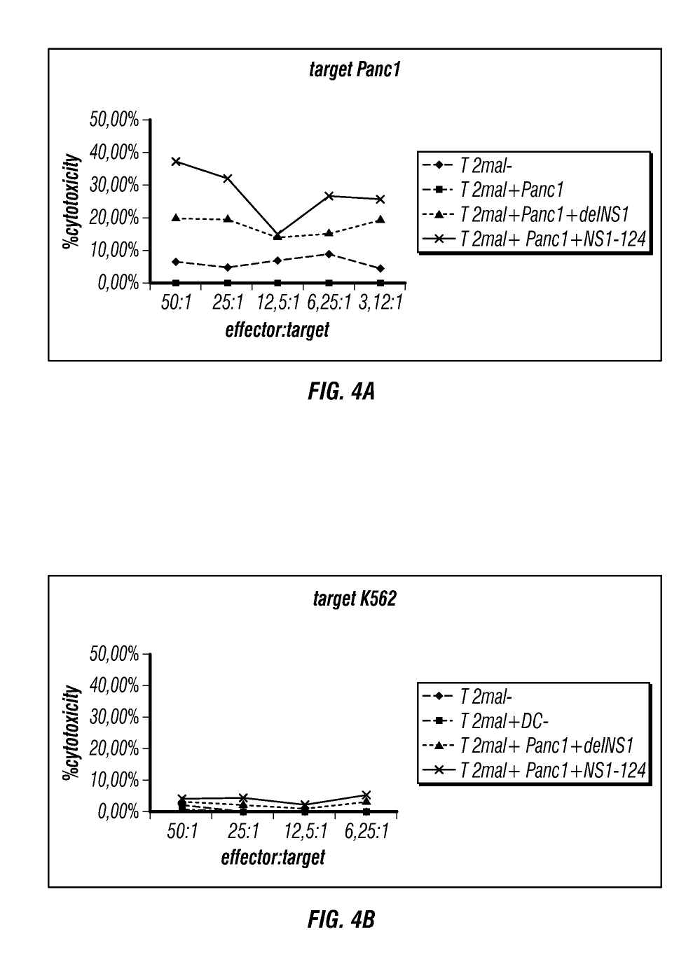

It was determined whether the viral infection of MODCs and the associated cytokines stimulation relates to an increase in the functional capacity of MODCs. As a functional assay the induction of a cytotoxic T-cell response by MODC, which were stimulated with lysate of a tumour cell lines, was used. Immature MODC were incubated with allogenic oncolysate generated from the Panc1 tumour cell line and subsequently infected either with delNS1 or with NS1-124 mutant virus. DCs were then incubated with allogenic oncolysate generated from the Panc1 tumour cell line. DCs were then co-cultured with autologous peripheral blood T-cells. No cytokines were added. The level of specific T-cell stimulation was then determined in a Europium assay against the specific target Panc-1. FIG. 4A shows a representative experiment out of 4 different donors. Virus-infected MODC were more potent to induce an immune response against the tumour cell line as compared to non-infected MODC. To rule out that observed cytotoxic effect on Panc1 cells was due to stimulated NK cells K562 were used as target (FIG. 4B). In this assay no cytotoxicity was observed.

### Example 6

### Virolysate Versus Oncolysate in the Capacity to Stimulate DCs

The delNS1 viruses and partial NS1 deletion viruses were shown to induce oncolysis in a murine tumour model. This observation rendered NS1 deletion mutant prototypes for oncolytic viruses therapeutic agent. It was determined, whether virolyses of tumour cells by a NS1 deletion virus is associated with an enhanced immunological capacity of the lysate to stimulated MODC as compared to tumour cell lyses generated in the absence of an immunostimulating agent.

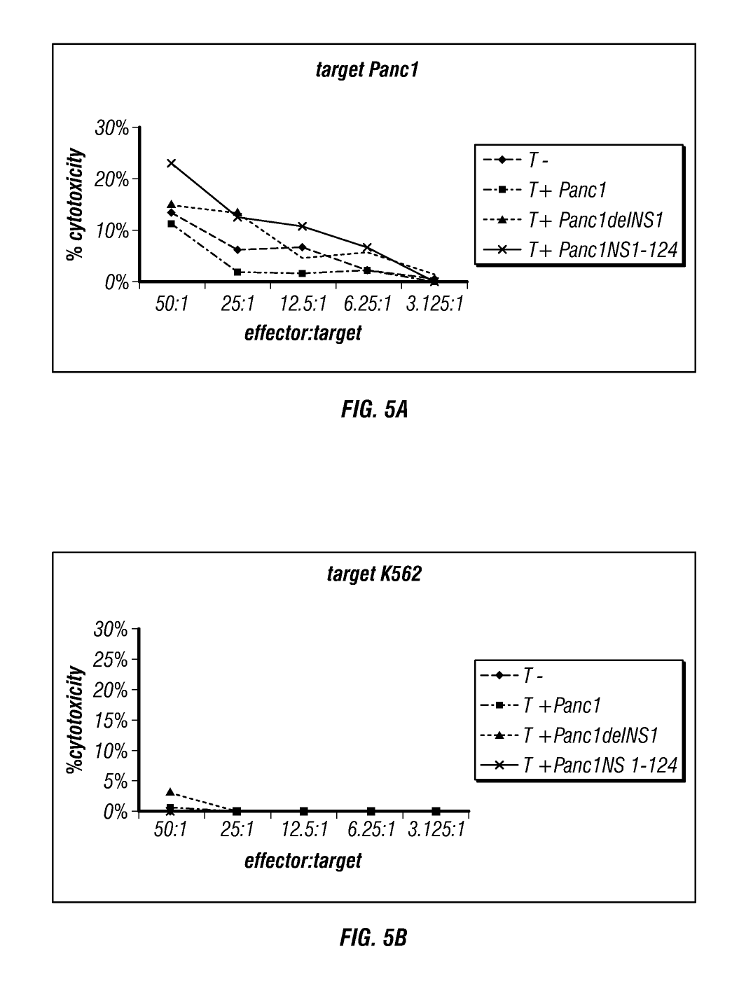

Immature monocyte derived DCs were incubated by virolysis using delNS1 or NS-124 or by oncolysate obtained by the freeze/thaw procedure. As a tumour cell line Panc1 was used. Lysate stimulated DCs were then co-cultured with autologous peripheral blood T-cells. No cytokines were added. The level of specific T-cell stimulation was then determined in an Europium assay against the specific target Panc-1. FIG. 5A shows one representative experiment out of 3 using 3 different donors for DC and T-cells. The virolysate had a tendency to stimulate DCs slightly better than the conventional oncolysate. However, the effect was not as pronounced as observed when dendritic cells were directly infected with the viruses. Again it was ruled out any NK mediated cell killing using K562 as targets (FIG. 4B). Moreover, cytometry showed no CD56 pos. cells again suggesting that NK cell did not contribute to the cytotoxic effect.

### Discussion

It is well known, that viruses induce a potent immune response to antigens expressed by the viral genome. These antigens can be endogenous viral antigens but also foreign antigens, which have been introduced into the viral genome by genetic engineering.

Here it is demonstrated that viral vaccine prototypes such as the influenza NS1 deletion viruses also have the capacity to enhance an immune response even when antigens are provided in trans and not expressed by the virus (in cis). In this way the NS1 deletion virus functions as an adjuvant like agent.

The enhancement of a CD8 restricted cytotoxic T-cell response by the virus was demonstrated using human dendritic cells. Induction of B-cells by the viral adjuvant was shown in human dendritic cells in a murine mouse model. This antigen specific immunostimulatory effect of the delNS1 virus is associated with a profound stimulation and activation of dendritic cells by the delNS1 virus as demonstrated by the induction of cytokines and activation makers. The activation of DCs is thought to be relevant for both, a T-cell and a B-cell immune response.

Interestingly this activation was not achieved by any of the viral components alone but by the whole virus. Single viral components could lead to some level of immune-stimulation such as isolated enhancement of activation markers (FIG. 3b), but were unable to induce the concerted array of viral DC stimulation (Tables 1-3). Therefore, the functional capacity of the DCs to the whole set of viral induced cytokines and activation markers but not single components is essential.

The data also demonstrates that DC related cytokine pattern depends on the presence of T-cells. Whereas virally induced DCs cells in the absence of T-cell mainly produce cytokines of the innate immune system, DC in the presence of T-cells produce polarising cytokines. In both settings virus infection greatly enhances the cytokine production. Since polarising cytokines, which were induced by the co-culture strongly favour a Th-1 response, NS1 deletion viruses are well prepared to induce a strong CTL-cell response and could act as a specific CTL immune enhance. Importantly cytokine stimulation by NS1 deletion viruses was enhanced as compared to wild type virus. This confirms that NS1 function as an immunosuppressive factor for the induction of the innate immune response. For example, infection of murine bone marrow derived DCs with the delNS1 virus lead to maturation of DCs and is associated with higher levels of NF-κB activation and the induction of the NF-κB dependent cytokines TNF, IL-6 and IL-1b as compared to wild type virus (Lopez et al., J Inf Dis, 2003). The higher induction of IFN-alpha by delNS1 virus as compared to wild type virus was also seen in human plasmocytoid DCs (Diebold et al., Nature 424: 324-328, 2003) and in LPS-induced or mature monocytic derived DCs (Efferson et al., J. Virol. 77: 7411-7424, 2003).

It is an important aspect of for clinical application, that the virus used according to the present invention is attenuated and shows the characteristics of vaccine strains in animal trials (Talon, J., Proc Natl Acad Sci USA., 97:4309-14, 2000). These properties suggest that application of an influenza virus with a deleted NS1 gene is feasible in humans. Previously it was shown that the immune-enhancing effect of the NS1 deletion viruses can be used for the induction of viral epitopes and chimeric epitopes expressed by the virus. The new aspect of the present invention is that the pro-inflammatory capacity of the virus can also be employed to enhance a immune response to foreign antigens which are processed in the virally infected cell but are not coded by the viral genome. This in trans stimulation renders the virus in a sense an adjuvant type of immunostimulation. Such an application would greatly broaden a possible clinical application of attenuated or replication defected viruses.

The methods of DC cultivation used in this assay have been used for vaccination based on whole tumour lysates in solid cancer. Our data now indicates that attenuated RNA viruses such as the NS1 deletion viruses might be a reasonable adjuvant to augment the effect of such DC based cancer vaccines. In this respect it is highly important that the NS1 deletion viruses are RNA viruses, which have the gene, which blocks the immunostimulating effect of the viral RNA deleted (Garcia-Sastre, A. Virology., 279:375-84, 2001. In this way more RNA is available for immunostimulation. This is beneficial for an anti cancer vaccination, since Leitner, W. et al. (Nat Med., 9:33-9, 2003) have shown, that the addition of dsRNA to a vaccine formula is able to overcome this self tolerance, effectively in a tumour animal model in vivo. Since most tumour-associated antigens are self antigens self tolerance is a major problem. Whereas pure DNA vaccination coding for an endogenous TAA was not effective, the combination of the DNA vaccination with an RNA replicon—generating dsRNA—could break the immunological tolerance towards the TAA and induced a protective immune response. The RNA replicon based tumour vaccine was associated with the activation PKR and RNAseL. Both of these proteins are major effector proteins within the type I IFN pathway. Therefore, the induction of a type I IFN response and PKR, as observed by NS1 deletion mutants, is a major step in the breakage of self tolerance.

Malignant tumours are a region of local immunosuppression, since malignant cancers themselves can produce immunosuppressive cytokines such as TGF or IL-10. Here we have demonstrated, that the infection of the malignant cell by the virus (FIG. 5) can enhance the immune response of stimulated DCs against tumour associated antigens. These data show, that a virus might overcome the tumour associated immunosuppression. In this way the delNS1 virus acts as a immunomodulating agent. Due to above mentioned properties of NS1 deletion viruses such as PKR induction and the high level of viral RNA such prototypes might be specifically valuable to exert such immunomodulating effects in cancer. Lately, it was shown that expression of dsRNA in a cell can also exert a similar effect. However, due to above mentioned data, again whole virus exerts a more profound effect than RNA alone. Moreover tumour cells can not easily be transduced with RNA in vivo. Therefore, a live virus might be substantial to exert this RNA based immune-enhancing effect in the clinical setting. Since delNS1 virus has even been shown to be able to induce lysis of susceptible tumour cells, such a viral prototype is ideal to antagonize tumour induced immunosuppression.

1-36. (canceled)

37. A pharmaceutical composition comprising an antigen and an adjuvant further defined as an apathogenic virus comprising a mutation, a truncation, a knock-out, or a reduced expression of an endogenous interferon antagonist, wherein the antigen is admixed with the apathogenic virus.

38. The composition of claim 37, wherein the apathogenic virus is an apathogenic vaccinia virus, adenovirus, hepatitis C virus, Newcastle disease virus, paramyxovirus, Sendai virus, respiratory syncytial virus, Filoviridae, herpes simplex virus type 1, reovirus, influenza virus or VSV.

39. The composition of claim 38, wherein the apathogenic virus is a reovirus, VSV or influenza virus.

40. The composition of claim 37, wherein the apathogenic virus is a genetically engineered virus selected from herpes virus Myb34.5, vaccinia virus MVA or Newcastle disease virus lacking the V protein.

41. The composition of claim 37, wherein the adjuvant is a genetically engineered influenza virus comprising a mutated or truncated NS1 protein, or a knockout or a reduced expression of the NS1 gene segment.

42. The composition of claim 41, wherein the expression of the NS1 protein is at least 5 fold lower compared to a wild type virus.

43. The composition of claim 42, wherein the expression of the NS1 protein is at least 10 fold lower compared to a wild type virus.

44. The composition of claim 41, wherein the reduction of NS1 expression is achieved by mutations in the 3′ terminal and/or 5′ non-coding nucleotides of the segment 8.

45. The composition of claim 44, wherein the reduction of NS1 expression is achieved by mutations in the NS1-ORF.

46. The composition of claim 45, wherein the reduction of NS1 expression is achieved by mutations by replacing the non-coding sequences of segment 8 with non-coding regions of the NA segment.

47. The composition of claim 41, wherein the genetically engineered influenza virus contains a deletion of the entire NS1 gene segment.

48. The composition of claim 41, wherein the genetically engineered influenza virus contains a truncated NS1 protein with a C-terminal deletion, while retaining the first 60 amino acids of the wildtype NS1 gene product.

49. The composition of claim 48, wherein the genetically engineered influenza virus contains a truncated NS1 protein with a C-terminal deletion, while retaining the first 80 amino acids of the wildtype NS1 gene product.

50. The composition of claim 49, wherein the genetically engineered influenza virus contains a truncated NS1 protein with a C-terminal deletion, while retaining the first 126 amino acids of the wildtype NS1 gene product.

51. The composition of claim 41, wherein the genetically engineered influenza virus contains the NS1-124 mutation, which only contains the N-terminal 124 amino acids of the NS1 protein.

52. The composition of claim 41, wherein the genetically engineered influenza virus contains the NS1-80 mutation, which only contains the N-terminal 80 amino acids of the NS1 protein.

53. The composition of claim 41, wherein the NS1 protein of the genetically engineered influenza virus lacks a functional RNA binding domain.

54. The composition of claim 41, wherein the adjuvant is a genetically engineered influenza virus, which is attenuated by replacing the non-coding sequences of the NS1 gene by those of other gene segments.

55. The composition of claim 41, wherein the influenza virus is an attenuated influenza A virus or attenuated influenza B virus.

56. The composition of claim 37, wherein the virus is an attenuated virus.

57. The composition of claim 37, comprising at least one additional adjuvant.

58. The composition of claim 57, wherein the at least one additional adjuvant is selected from mineral gels, aluminum hydroxide, surface active substances, lysolecithin, pluronic polyols, polyanions or oil emulsions, or a combination thereof.

59. The composition of claim 37, further comprising buffer substances.

60. The composition of claim 37, comprising a pharmaceutically acceptable carrier.

61. The composition of claim 37, wherein the antigen is selected from tumor antigens or antigens of infectious pathogens like different viruses, bacteria, parasites or fungi.

62. The composition of claim 37, wherein the antigen is gp160, gp120 or gp41 of HIV, HA, and NA of influenza virus, an antigen of an endogenous retrovirus, an antigen of human papilloma virus, melanoma gp100, survivin, Her2neu, NY-ESO, a tuberculosis antigen, a hepatitis antigen, or a polio antigen.

63. The composition of claim 62, wherein the antigen is E6 or E7 protein.

64. The composition of claim 37, further defined as comprising a cytokine.

65. The composition of claim 37, wherein the virus comprises a genetic sequence for an immunostimulatory cytokine.

66. The composition of claim 37, further defined as comprised in a pharmaceutically acceptable carrier.

67. The composition of claim 37, further defined as comprised in a pharmaceutically acceptable carrier adapted for intranasal delivery in the form of drops or a spray.

68. The composition of claim 37, further defined as comprised in a pharmaceutically acceptable carrier adapted for subcutaneous, intramuscular, intravascular or intraperitoneal injection and comprising a stabilizing carrier.

69. A method of inducing an immune-enhancing effect of the antigen or overcoming pathogen induced immunosuppression or cancer induced immunosuppression comprising:
obtaining a composition of claim 37; and
administering the composition to a subject;
wherein an immune-enhancing effect of the antigen is induced in the subject and/or pathogen and/or cancer induced immunosuppression is overcome in the subject.

70. A method of in vitro activation of dendritic cells with a specific antigen comprising contacting dendritic cells in vitro with a composition of claim 37 comprising said antigen.

71. The method of claim 70, wherein the dendritic cells are contacted with the composition for 10 minutes to 8 hours.

72. The method of claim 71, wherein the dendritic cells are contacted with the composition for 10 to 60 minutes.

73. The method of claim 70, wherein the specific antigen is an isolated tumor or virus antigen, a recombinant tumor or virus antigen or a tumor or virus lysate.

74. The method of claim 73, wherein virus lysate is obtained through infection of tumor cells with the apathogenic virus.

75. The method of claim 74, wherein virus lysate is obtained through infection of tumor cells with an NS1 deficient influenza virus.

76. A method of inducing an immune-enhancing effect of an antigen comprising:
obtaining an antigen;
obtaining admixed with an apathogenic virus comprising a mutation, a truncation, a knock-out, or a reduced expression of an endogenous interferon antagonist; and
administering the antigen and apathogenic virus to a subject;
wherein an immune-enhancing effect of the antigen is induced in the subject.

77. A pharmaceutical composition comprising an antigen and an adjuvant further defined as an influenza virus having no active interferon antagonist.
